(19) United States
(12) Patent Application Publication
(10) Pub. No.: US 2014/0328939 A1
(43) Pub. Date: Nov. 6, 2014

(54) METHOD FOR PREPARING BIOCOMPATIBLE SMALL INTESTINAL MUCOSA HYDROGEL CAPABLE OF CONTROLLING IN-VIVO DEGRADATION PERIOD

(71) Applicants: Da Yeon Kim, Gyeonggi-do (KR); Moon Suk Kim, Gyeonggi-do (KR); So Mi Yoon, Busan (KR); Bit Na Lee, Seoul (KR)

(72) Inventors: Da Yeon Kim, Gyeonggi-do (KR); Moon Suk Kim, Gyeonggi-do (KR); So Mi Yoon, Busan (KR); Bit Na Lee, Seoul (KR)

(73) Assignee: AJOU UNIVERSITY INDUSTRY-ACADEMIC COOPERATION FOUNDATION, Suwon-si, Gyeonggi-do (KR)

(21) Appl. No.: 14/343,629

(22) PCT Filed: Apr. 2, 2012
(86) PCT No.: PCT/KR2012/002455

§ 371 Date: Jul. 1, 2014

(30) Foreign Application Priority Data
Sep. 9, 2011 (KR) 10-2011-0092202

(51) Int. Cl. A61K 35/38; A61K 9/06

(52) U.S. Cl. CPC A61K 35/38; A61K 9/06
USPC 424/551

(57) ABSTRACT

The present invention relates to a method for preparing a biocompatible small intestinal submucosa hydrogel with a controllable in-vivo degradation period, more particularly to a method for preparing a small intestinal submucosa hydrogel which is formed in a solution phase by forming chemical crosslinkages by solubilizing a biocompatible small intestinal submucosa powder and allows the degradation period of a gel formed during in-vivo injection by controlling the degree of crosslinking and a method for preparing a small intestine submucosa drug carrier or a small intestine submucosa support using the hydrogel.

### TECHNICAL FIELD

The present invention relates to a method for preparing a biocompatible small intestinal submucosa hydrogel with a controllable in-vivo degradation period, more particularly to a method for preparing a small intestinal submucosa hydrogel which is formed in a solution phase by forming chemical crosslinkages by solubilizing a biocompatible small intestinal submucosa powder and allows the degradation period of a gel formed during in-vivo injection by controlling the degree of crosslinking and a method for preparing a small intestine submucosa drug carrier or a small intestine submucosa support using the hydrogel.

### BACKGROUND ART

Recently, stem cells have been drawing much attention in the field of regenerative medicine. Numerous research has confirmed the capabilities of stem cells to be differentiated into various tissues under the action and control of various cytokines. Under this background, it is expected that various artificial organs such as cartilage, bones, blood vessels, etc., can be developed by integrating cells, genes and stem cells with stimulus-sensitive hydrogels.

Natural materials derived from natural products, animals or humans have very superior biocompatibility. A typical example is the extracellular matrix (ECM) which is extracted from humans or animals and is capable of regulating cellular function. Since a support prepared from a natural material induces less inflammatory response and can provide excellent biofunctionality, biodegradability, etc., after implantation, the natural material is viewed as an ideal material for a support for tissue engineering.

Among the natural materials, pig small intestine submucosa may be used as a support for regenerating damaged tissues. Although bone marrow transplantation or donated organs such as heart, kidneys, eyes, etc., are used to heal the organs damaged due to various diseases, demand far outstrips supply. Thus, development of artificial organs using tissue engineering is required. Tissue engineering is a field aimed at restoration or maintenance of functions of lost organs using biological substitutes based on the principles of life science and bioengineering requiring interdisciplinary cooperations.

Pig small intestine submucosa is a tissue without cells which is nearly free from immune response. Collagen I and II found in skin account for more than 90% and collagen V, VI, etc., are also present in small quantities. Also, the small intestine submucosa contains extracellular matrices (ECMs) such as glycosaminoglycan, fibronectin, chondroitin sulfate, heparin, heparin sulfate and hyaluronic acid and various cytokines such as basic fibroblast growth factor (FGF2), nerve growth factor (NGF), transforming growth factor β (TGF-β), epidermal growth factor (EGF), vascular endothelial growth factor (VEGF), insulin-like growth factor 1 (IGF-1), etc., in large quantities.

Since the small intestine submucosa contains ECMs and cytokines, it can functionally help the adhesion, growth, migration, differentiation, etc., of cells and can be used in various applications including tissue regeneration.

The small intestine submucosa was first studied by Badylak at Purdue University in the USA and is studied by many researchers including the Cook Group. As a result of these researches, vascular (veinous or arterial), dermal, epithelial and skeletal implantation, bile duct regeneration, bladder implantation for treating incontinence, or the like are available at present. As such, the small intestine submucosa can be used in various applications as biological substitutes and needs a further study for its use as a biocompatible material.

However, mechanical or chemical treatment is necessary to improve the properties of the natural material.

Recently, an injectable hydrogel has been drawing much attention in the field of medicine. It is expected to be widely applicable from a medical filler to a system for releasing a physiologically active substance, organ/tissue regeneration based on a 3-dimensional structure, etc. The injectable hydrogel is advantageous in that it can be injected by simply using, for example, a syringe without a surgical operation. The injectable hydrogel can be injected using a syringe since it is fluid-like ex vivo in general, and gelation occurs once injected in vivo. That is, once it is implanted, it can serve as a delivery system for sustained release of a drug or a physiologically active substance or as a support for cellular growth.

However, there are limitations in using the hydrogel for drug delivery systems or tissue engineering because the control of degradation period is difficult. Accordingly, it is important to prepare a hydrogel whose degradation period can be suitably controlled according to the intended purposes.

### DISCLOSURE

### Technical Problem

The present invention is directed to providing a method for preparing a small intestinal submucosa hydrogel, by crosslinking small intestine submucosa which contains ECMs and cytokines that help cellular functions such as adhesion, growth, migration, differentiation, etc., and thus can be applied in many fields including tissue regeneration or wound healing using a crosslinking agent, thereby forming a gel which is not easily degradable and whose degradation period is controllable with the concentration of the crosslinking agent.

### Technical Solution

In an aspect, the present invention provides a method for preparing a small intestinal submucosa hydrogel, including chemically crosslinking small intestine submucosa of an animal using a chemical crosslinking agent.

In another aspect, the present invention provides a method for preparing a small intestine submucosa drug carrier or support, including injecting a small intestinal submucosa hydrogel prepared by the above-described method in vivo and forming a gel.

### Advantageous Effects

A biocompatible small intestinal submucosa hydrogel according to the present invention is advantageous in that degradation time can be controlled with the concentration of a crosslinking agent and degradation period can be controlled with the degree of crosslinkage.

In addition, the small intestinal submucosa hydrogel according to the present invention is advantageous in that it can be injected to be used as a drug carrier or a support for tissue engineering.

### BRIEF DESCRIPTION OF DRAWINGS

FIG. 1 shows photographic images illustrating a small intestinal submucosa hydrogel prepared in Example 1 and its gelation after subcutaneous injection into a rat.

FIG. 2 shows photographic images illustrating gels removed from a rat at different times after subcutaneous injection of a small intestinal submucosa hydrogel prepared in Example 1 into a rat.

FIG. 3 shows SEM images illustrating cross-sections of gels extracted 1 week after subcutaneous injection of a small intestinal submucosa hydrogel prepared in Example 1 into a rat.

FIG. 4 shows the size (volume) of gels measured at different times after subcutaneous injection of a small intestinal submucosa hydrogel prepared in Example 1 into a rat.

FIG. 5 shows H&E staining images of gels extracted at different times after subcutaneous injection of a small intestinal submucosa hydrogel prepared in Example 1 into a rat.

FIG. 6 shows ED1 immunofluorescence staining images of gels extracted at different times after subcutaneous injection of a small intestinal submucosa hydrogel prepared in Example 1 into a rat.

FIG. 7 shows the change in albumin concentration in the blood of a rat with time measured in Test Example 3.

FIG. 8 shows safranin O staining images of gels extracted after a predetermined amount of time after injection of a small intestinal submucosa hydrogel with chondrocytes in Test Example 4.

### BEST MODE FOR CARRYING OUT INVENTION

Hereinafter, the present invention is described in more detail.

A method for preparing a small intestinal submucosa hydrogel according to the present invention includes chemically crosslinking the small intestine submucosa of an animal, specifically a mammal, more specifically a mammal excluding humans, using a chemical crosslinking agent.

Since the small intestine submucosa used in the present invention contains extracellular matrices (ECMs) and cytokines, it can functionally help the adhesion, growth, migration, differentiation, etc., of cells and can be used in various applications including tissue regeneration.

To describe in detail, the method for preparing a small intestinal submucosa hydrogel according to the present invention includes:

(1) preparing a small intestine submucosa solution by mixing small intestine submucosa with an acidic solution and pepsin;

(2) adjusting the pH of the solution obtained in (1) to a biologically compatible pH using an alkaline solution;

(3) preparing a powder by freeze-drying the solution obtained in (2); and

(4) solubilizing the powder obtained in (3) and crosslinking the same by mixing with a chemical crosslinking agent.

First, the small intestine submucosa of a mammal is separated. The jejunum is removed from the small intestine of a mammal and immersed in a physiological saline and then in a phosphate buffered saline. After removing the mesentery, the small intestine is cut to a predetermined size. solution. After turning the inside of the cut-out small intestine out, the mucosa is removed by mechanical rubbing. Subsequently, the small intestine is inverted again and the small intestine submucosa is separated by removing the serosa and the muscularis.

The separated small intestine submucosa is freeze-dried and ground. The ground small intestine submucosa is mixed with an acidic solution and pepsin. The mixture solution is adjusted to pH 5.5-7.8, specifically to pH 6.5-7.5, using an alkaline solution to prepare a biocompatible small intestinal submucosa powder that can be formed into a gel.

The acidic solution may be a solution of pH 2.5-4.5. Specifically, an aqueous solution containing 1-5 wt % of an acid selected from acetic acid, p-toluenesulfonic acid and maleic acid may be used.

The alkaline solution is used to neutralize the acidity of the acidic solution and reduce inflammatory responses of nearby cells when the small intestinal submucosa hydrogel of the present invention is subcutaneously injected or used as a support for tissue engineering. For example, one or more selected from sodium hydroxide (NaOH), sodium carbonate (Na₂CO₃), sodium bicarbonate (NaHCO₃), disodium hydrogen phosphate (Na₂HPO₄), calcium bicarbonate (Ca(HCO₃)₂), calcium hydroxide (Ca(OH)₂), calcium hydroxide nitrate (Ca(OH)NO₃), calcium hydroxide chloride (Ca(OH)CI), calcium hydroxide cyanide (Ca(OH)CN), potassium hydroxide (KOH), ammonium hydroxide (NH₄OH) and sodium acetate (CH₃COONa) may be used. The alkaline solution is used to adjust pH to 5.5-7.8, similarly to that in vivo.

Since the small intestine submucosa solution is easily degraded by the body fluid after in-vivo injection, it may be crosslinked to increase the period of time during which it remains as a gel and to control its degradation period. The chemical crosslinking agent serves to form crosslinkages between polymer chains, thereby suppressing degradation by the body fluid and allowing control of degradation period. The mechanical properties of a support prepared from a natural material can be improved through chemical treatment using, for example, a crosslinking agent.

The chemical crosslinking agent that can be used in the present invention may be selected from an aldehyde-based compound, a water-soluble carbodiimide-based compound, an epoxy compound and a diisocyanate compound. More specifically, the water-soluble carbodiimide-based compound may be 1-ethyl-3-(3-dimethylaminopropyl)carbodiimide, dicyclohexyl carbodiimide or 1-ethyl-3-(2-morpholinyl-4-ethyl)carbodiimide and the aldehyde-based compound may be, for example, formaldehyde, glutaraldehyde, dextrin aldehyde, etc. Most specifically, 1-ethyl-3-(3-dimethylaminopropyl)carbodiimide (EDC) may be used.

In the present invention, the concentration of the crosslinking agent in the crosslinking reaction may be from 0.001 mM to 1 M, more specifically 0.01-100 mM, most specifically 0.1-10 mM.

The present invention also provides a method for preparing a small intestine submucosa drug carrier or support, including injecting the small intestinal submucosa hydrogel prepared by the above-described method in vivo and forming a gel. The small intestine submucosa hydrogel according to the present invention, which is prepared as an aqueous solution by forming chemical crosslinkages using the crosslinking agent and forms a gel in situ after in-vivo injection, is an injectable hydrogel and can be used as an injectable drug carrier or a support for tissue engineering.

The present invention will be described in more detail through examples. However, the scope of the present invention is not limited by the examples.

### EXAMPLES 1-31

### (1) Separation of Small Intestine Submucosa from Pig and Storage Thereof

Adipose tissue was removed first from the jejunum of a pig within 4 hours after death. The jejunum was washed cleanly with water and cut to a length of about 10 cm and then washed with physiological saline. After removing the compact outer layer of the cut jejunum by applying physical force, the jejunum was inverted and the muscularis was removed and only the small intestine submucosa was separated. The small intestine submucosa was washed with physiological saline and stored in a cryogenic refrigerator at −80° C.

### (2) Preparation of Small Intestine Submucosa Powder

The small intestine submucosa stored at −80° C. in (1) was freeze-dried and prepared into a powder of 10-30 μm size using a freezer mill (6700, SPEX Inc., USA).

### (3) Preparation of Biocompatible Small Intestinal Submucosa Powder

1 wt % of the small intestine submucosa in powder form was mixed with distilled water containing 3% acetic acid and 0.1% pepsin by stirring at room temperature for 48 hours. The resulting small intestine submucosa solution was adjusted to pH 7.4 using 1 N sodium hydroxide (NaOH). The pH-adjusted solution was freeze-dried and prepared into a biocompatible small intestinal submucosa powder through freezer grinding.

### (4) Crosslinking using Chemical Crosslinking Agent

The prepared small intestinal submucosa powder was dissolved in phosphate buffered saline to 15 wt % in a 5-mL vial. Then, after adding 0.1 mM (Example 1), 1.0 mM (Example 2) or 10 mM (Example 3) of 1-ethyl-3-(3-dimethylaminopropyl)carbodiimide (EDC) as a crosslinking agent, the mixture was stirred at room temperature for 24 hours. As a result of the crosslinking of the small intestine submucosa solution, an injectable hydrogel was obtained.

### COMPARATIVE EXAMPLE 11

The small intestinal submucosa powder prepared in (3) of Example 1 was dissolved in phosphate buffered saline to 15 wt % to prepare an injectable.

### Test Example 1. In-vivo Injection of Crosslinked Small Intestinal Submucosa Powder

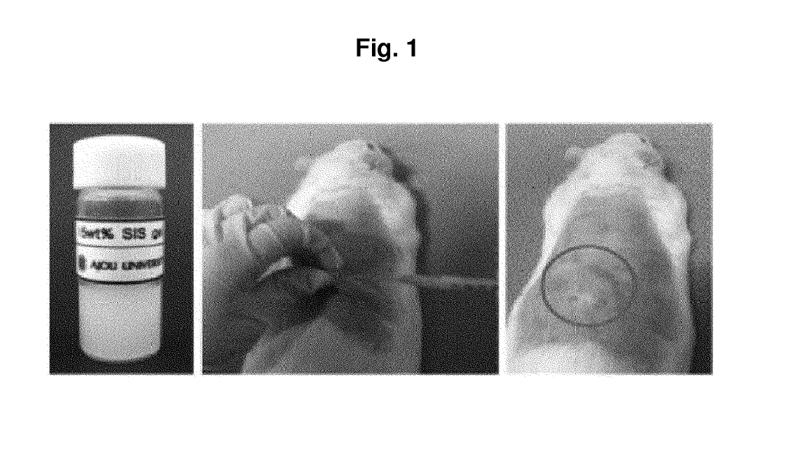

The small intestinal submucosa powders prepared in Examples 1-3 were sterilized with ethylene oxide gas and injected in vivo as injectables (FIG. 1).

1 mL of the hydrogel solutions prepared in Examples 1-3 were subcutaneously injected to SD rats (male, 8 weeks old, 350-380 g) using a syringe. For comparison, the small intestine submucosa solution without containing a crosslinking agent obtained in Comparative Example 1 was subcutaneously injected to an SD rat.

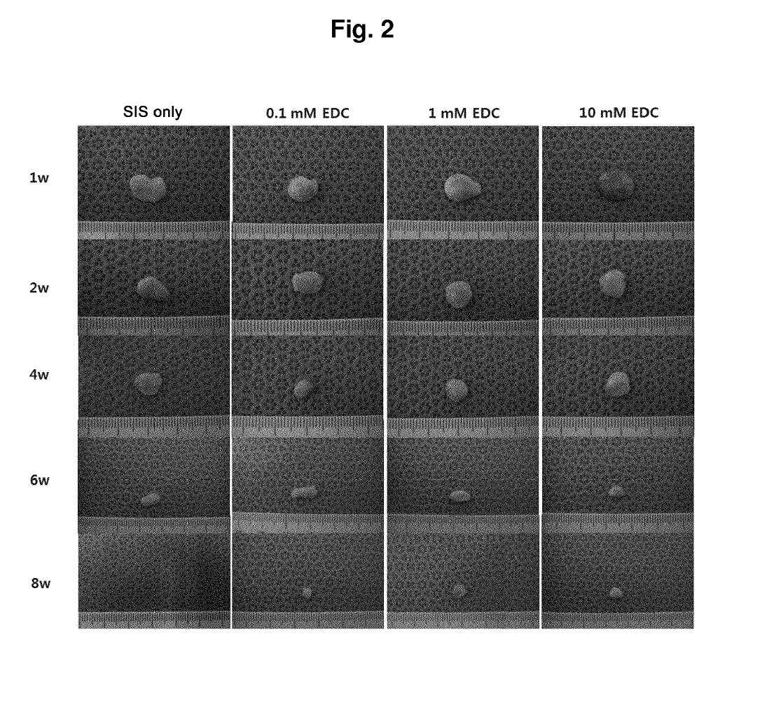

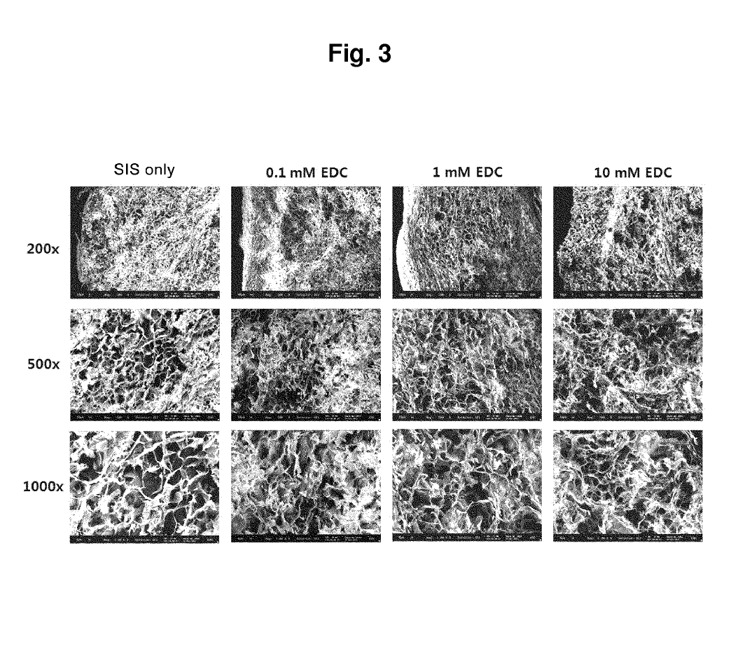

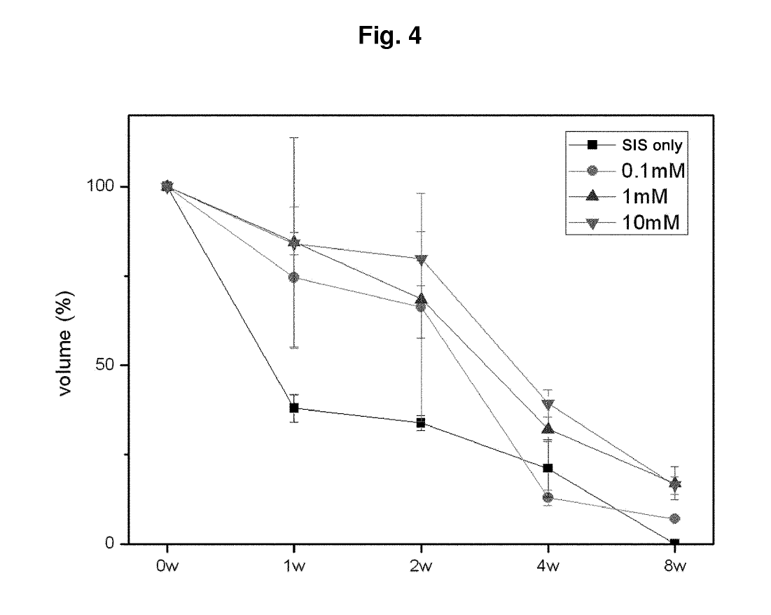

It was observed that hard gels were formed immediately after the injection. The formed gels were extracted 1, 2, 4, 6 and 8 weeks after the injection to investigate the degree of degradation (FIG. 2). Also, the cross-sections of the gels were observed by scanning electron microscopy (SEM) 1 week after the injection (FIG. 3). The crosslinked small intestine submucosa solutions exhibited maintenance of harder gels and slower degradation of gels as compared to the uncrosslinked small intestine submucosa solution (FIG. 4). This demonstrates that crosslinkages were formed by the crosslinking agent and the gel degradation rate can be controlled by crosslinking the small intestine submucosa solutions.

### Test Example 2. Histological Evaluation of Small Intestine Submucosa Gel

The gels formed in Example 1 were extracted 1, 2 and 4 weeks after the injection, fixed in 10% formalin solution and prepared into paraffin blocks. The paraffin blocks were sliced into 4-μm sections and stained with H&E and ED1 for histological evaluation.

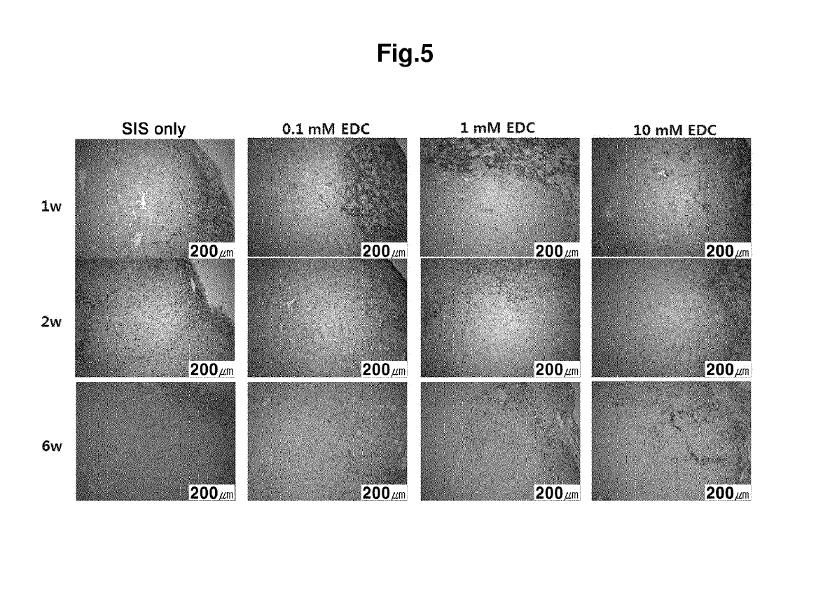

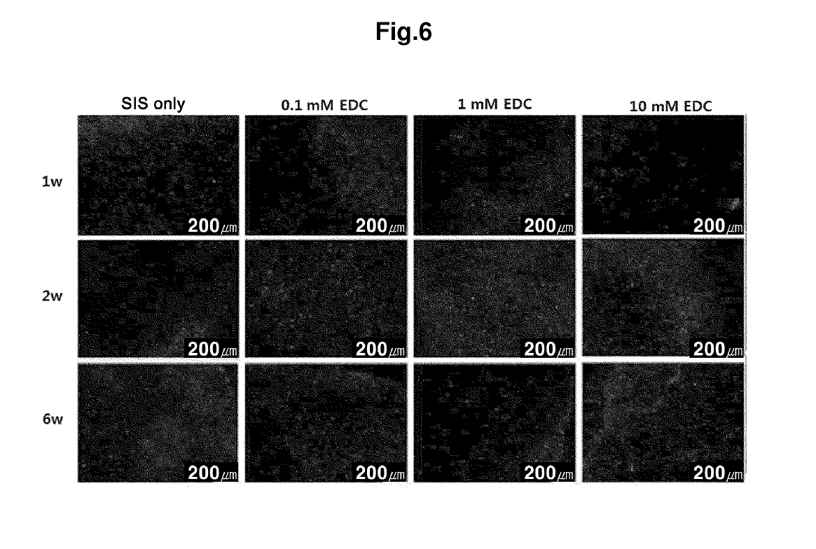

H&E staining is the most used staining method using hematoxylin which specifically stains the cell nucleus and eosin which stains the cytoplasm. Because it provides information on both the nucleus and the cytoplasm, H&E staining was conducted to observe cellular behavior and morphology after the injection (FIG. 5). Also, the expression of ED1 (mouse anti-rat CD68; Serotec, UK) was investigate to confirm the inflammatory response of the injected hydrogels (FIG. 6). As a result, it was confirmed that the hydrogels of the present invention are biocompatible and do not induce inflammatory response and thus it can be used as a support for tissue engineering.

### Test Example 3. In-vivo Drug Release Test

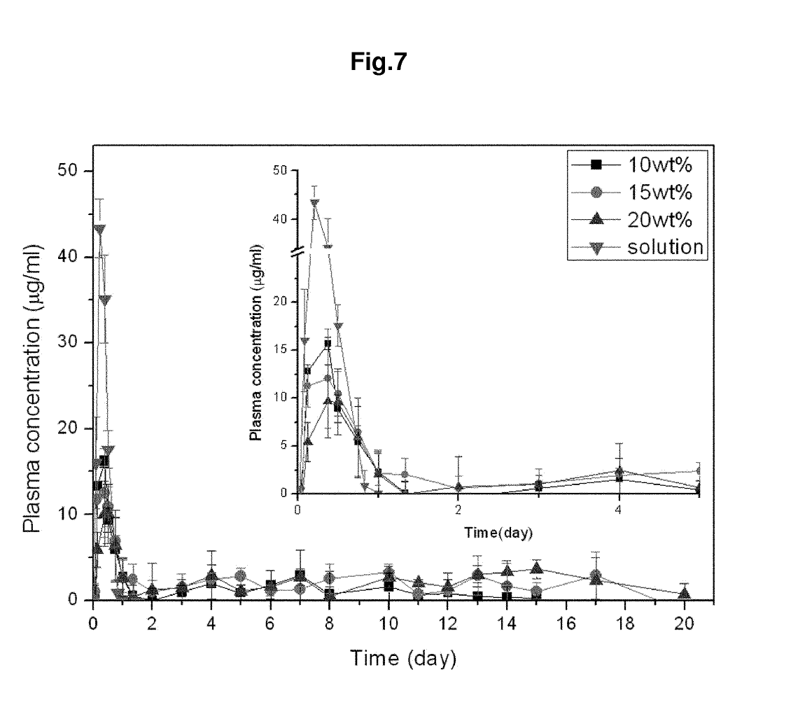

To demonstrate the potentiality as a drug carrier, the small intestine submucosa solution prepared in Example 1 was mixed with a drug and in-vivo release was monitored (FIG. 7). The prepared small intestinal submucosa powder was dissolved in phosphate buffered saline to 10, 15 and 20 wt %, respectively, to prepare homogeneous solutions. Then, FITC-labeled albumin (FITC-BSA) was added to a concentration of 1 mg/mL. 0.5 mL of the albumin-containing small intestine submucosa solutions were subcutaneously injected to nine SD rats (male, 8 weeks old, 350-380 g) using a syringe (21 G, 1-cc needle). For comparison, 0.5 mL of a solution containing FITC-labeled albumin of the same concentration was subcutaneously injected to three rats. When only albumin was subcutaneously injected, the drug was not released any more after 1 day. In contrast, in case of the albumin-containing small intestinal submucosa hydrogel, the drug was continuously released for about 9 days. When the gels were extracted from the rats and subjected to concentration measurement, the drug was found to be continuously released up to 20 days depending on the concentration of the small intestine submucosa hydrogel.

### Test Example 4. In-vivo Chondrocyte Delivery Test

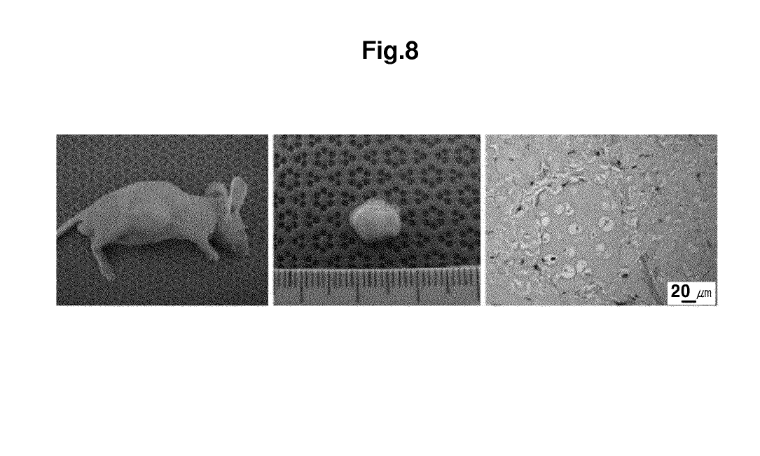

To demonstrate the potentiality as a cell delivery system, the small intestine submucosa solution prepared in Example 1 was mixed with chondrocytes and differentiation of chondrocytes in vivo was monitored (FIG. 8). The prepared small intestinal submucosa powder was dissolved in phosphate buffered saline to 15 wt % to prepare a homogeneous solution. Then, chondrocytes were added at a concentration of 2×10⁶ cells/mL. 0.5 mL of the chondrocytes-containing small intestine submucosa solution was subcutaneously injected to a nude mouse (male, 6 weeks old) using a syringe (21 G, 1-cc needle). Through extraction and safranin O staining, it was confirmed that the injected chondrocytes proliferated and differentiated in the small intestine submucosa gels.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for preparing a small intestinal submucosa hydrogel, comprising chemically crosslinking small intestine submucosa of an animal excluding humans using a chemical crosslinking agent.

2. The method for preparing a small intestinal submucosa hydrogel according to claim 1, which comprises:
(1) preparing a small intestine submucosa solution by mixing small intestine submucosa with an acidic solution and pepsin;
(2) adjusting the pH of the solution obtained in (1) to a biologically compatible pH using an alkaline solution;
(3) preparing a powder by freeze-drying the solution obtained in (2); and
(4) solubilizing the powder obtained in (3) and crosslinking the same by mixing with a chemical crosslinking agent.

3. The method for preparing a small intestinal submucosa hydrogel according to claim 1, wherein the chemical crosslinking agent is selected from an aldehyde-based compound, a water-soluble carbodiimide-based compound, an epoxy compound and a diisocyanate compound.

4. The method for preparing a small intestinal submucosa hydrogel according to claim 3, wherein the chemical crosslinking agent is 1-ethyl-3-(3-dimethylaminopropyl)carbodiimide, formaldehyde, glutaraldehyde, dextrin aldehyde or dicyclohexylcarbodiimide, 1-ethyl-3-(2-morpholinyl-4-ethyl) carbodiimide.

5. The method for preparing a small intestinal submucosa hydrogel according to claim 2, wherein the biologically compatible pH in (2) is pH 5.5-7.8.

6. The method for preparing a small intestinal submucosa hydrogel according to claim 2, wherein the concentration of the crosslinking agent in (4) is from 0.001 mM to 1 M.

7. A method for preparing a small intestine submucosa drug carrier or support, comprising injecting a small intestinal submucosa hydrogel prepared by the method according to claim 1 in vivo and forming a gel.
